(19) United States
(12) Patent Application Publication
(10) Pub. No.: US 2024/0428891 A1
(43) Pub. Date: Dec. 26, 2024

(54) MOLECULE DETECTIONS

(71) Applicant: Hewlett-Packard Development Company, L.P., Spring, TX (US)

(72) Inventors: Anton Wiranata, Boise, ID (US); Yiming Zuo, Palo Alto, CA (US); Paul J. Benning, Corvallis, OR (US); Brian John Keefe, San Diego, CA (US); Amy Devitt, Palo Alto, CA (US)

(73) Assignee: Hewlett-Packard Development Company, L.P., Spring, TX (US)

(21) Appl. No.: 18/684,133

(22) PCT Filed: Aug. 25, 2021
(86) PCT No.: PCT/US2021/047590

§ 371 Date: Feb. 15, 2024

(51) Int. Cl. G16B 40/20; G01N 21/64; G16B 45/00

(52) U.S. Cl. CPC G16B 40/20; G01N 21/6428; G16B 45/00; G01N 2021/6441

(57) ABSTRACT

Examples of methods are described herein. In some examples, a method includes generating a plot of a fluorescence signal over time. In some examples, the fluorescence signal is measured from a substance. In some examples, the method includes detecting, using a machine learning model, a target molecule in the substance based on the plot.

### BACKGROUND

Molecules are groups of atoms. Examples of molecules include nucleic acids. Nucleic acids are molecular structures made from polynucleotide chains, each containing a five-carbon sugar backbone, a phosphate group, and a nitrogen base. Ribonucleic acid (RNA) is a nucleic acid (e.g., molecular structure) that may include a single polynucleotide chain. Deoxyribonucleic acid (DNA) is a nucleic acid (e.g., molecular structure) including two polynucleotide chains that form a double helix. DNA and RNA include a sequence of nucleobase pairs (of four nucleobases cytosine, guanine, adenine, and thymine). For example, DNA includes nucleobases between sugar-phosphate backbones of the double helix. DNA and/or RNA serve as genetic instructions for the reproduction of organisms and viruses. Different organisms and viruses include different nucleic acid strands. For instance, different viruses may include different RNA strands.

### BRIEF DESCRIPTION OF THE DRAWINGS

FIG. 1 is a flow diagram illustrating an example of a method for target molecule detection;

FIG. 2 is a block diagram illustrating an example of engines that may be utilized in accordance with some examples of the techniques described herein;

FIG. 3 is a block diagram of an example of an apparatus that may be used in molecule detection;

FIG. 4 is a block diagram illustrating an example of a computer-readable medium for molecule detection;

FIG. 5A is a graph illustrating examples of a first plot of a first fluorescence signal and a second plot of a second fluorescence signal;

FIG. 5B is a graph illustrating examples of a first plot of a first fluorescence signal and a second plot of a second fluorescence signal;

FIG. 5C is a graph illustrating examples of a first plot of a first fluorescence signal and a second plot of a second fluorescence signal; and

FIG. 6 is a diagram illustrating an example of an architecture of a machine learning model that may be utilized in some examples of the techniques described herein.

### DETAILED DESCRIPTION

Examples of the techniques described herein provide approaches for the detection of a target molecule in a substance. A target molecule is a molecule to be detected. An example of a target molecule is a nucleic acid strand. For instance, a target molecule may be a nucleic acid strand that identifies a specific virus, bacteria, or organism. A nucleic acid strand is a portion of DNA and/or RNA. A substance is physical matter. For example, a substance may potentially include a target molecule. An example of a substance is a nucleic acid sample with or without other physical matter. A nucleic acid sample is biological material including a nucleic acid (e.g., DNA and/or RNA). Examples of nucleic acid samples include saliva, blood, mucus, sputum, urine, stool, cells, tissue, skin, etc. Some examples of the techniques described herein may be utilized to detect a nucleic acid strand in a nucleic acid sample.

An amplification procedure is a procedure to replicate or “amplify” a nucleic acid strand. Examples of amplification procedures include quantitative polymerase chain reaction (qPCR), pulse-controlled amplification (PCA), and reverse transcriptase polymerase chain reaction (RT-PCR). In an amplification procedure, a nucleic acid sample is repeatedly heated and cooled. Throughout heating and cooling cycles, measurements (e.g., fluorescence measurements) are taken.

In some examples of amplification procedures, a primer and/or fluorophore is added to a nucleic acid sample. For instance, a substance may include a nucleic acid sample, a primer, and/or a fluorophore. A primer is a molecule that binds to a target nucleic acid strand (e.g., to a beginning and/or end of a target nucleic acid strand). For example, a nucleic acid sample may be heated (e.g., heated to 94° Celsius (C.)) or another temperature) to denature the nucleic acid (e.g., open the nucleobase pairs to expose the nucleobases). In some examples, after denaturing the nucleic acid, the nucleic acid sample may be cooled (e.g., cooled to between 50-60° (C.) or another temperature, annealed, etc.) and the primer may bind with the beginning and/or end of a target nucleic acid strand. In some examples, after cooling, the nucleic acid sample may be warmed (e.g., warmed to 72° C. or another temperature) and an enzyme (e.g., polymerase) may replicate the target nucleic acid strand (e.g., may add bases to the target nucleic acid strand from the primer binding site(s)). A fluorophore is a chemical compound that emits light after excitation. For example, a fluorophore may bond with a target nucleic acid strand. The nucleic acid sample may be excited with light. For example, a light emitting diode (LED), laser, or xenon lamp may be utilized to excite the nucleic acid sample with ultraviolet light and/or visible light, etc. The bonded fluorophore may emit light after excitation. The emitted light may be measured with a detector (e.g., light sensor, camera, etc.). For instance, the detector may produce a fluorescence measurement of the nucleic acid sample. Multiple cycles (e.g., denaturing, annealing, replication, and/or measurement) of the amplification procedure may be performed to create additional copies and measure an amount of the target nucleic acid strand in the nucleic acid sample. For instance, a fluorescence measurement may increase as additional copies are created.

Some examples of fluorophores may include 6-carboxyfluorescein (FAM), Cy5™, hexachlorofluorescein (HEX), and Texas Red™ (TEX). The wavelength of excitation light and/or emitted light utilized may vary in accordance with the fluorophore utilized. Examples of wavelengths for excitation light may include 495 nanometers (nm) for FAM, 648 nm for Cy5, 538 nm for HEX, and 596 nm for TEX. Examples of wavelengths for emitted light (e.g., detected light) may include 520 nm for FAM, 668 nm for Cy5, 555 nm for HEX, and 613 nm for TEX. In some examples, another fluorophore or fluorophores with a corresponding wavelength or wavelengths may be utilized. In some examples, the wavelength of excitation light provided and/or emitted light detected may vary from the examples given and/or may be performed over wavelength ranges. In some examples, one fluorophore with an excitation light wavelength (or wavelength range) and an emitted (e.g., detected) light wavelength (or wavelength range) may be utilized. In some examples, a combination of (e.g., 2, 3, 4, 5, 6, etc.) fluorophores, excitation light wavelengths, and/or emitted light (e.g., detected light) wavelengths may be utilized. For instance, a combination of FAM and Cy5 fluorophores with corresponding wavelengths may be utilized. In some examples, a fluorescence measurement for multiple fluorophores may be a sum, average, maximum, or other combination or selection of individual metrics (e.g., voltages, currents, etc.) for the respective fluorophores and/or wavelengths. In some examples, fluorescence measurements for multiple fluorophores may be taken individually (e.g., concurrently, in parallel, etc.) with respective fluorophores and/or wavelengths to produce respective measurements. In some examples, a single reaction chamber or multiple reaction chambers may be utilized in accordance with some examples of the techniques described herein.

PCA may be utilized to replicate and/or measure a target nucleic acid strand (e.g., target nucleic acids) from pathogens such as bacteria and viruses. For instance, PCA may be utilized to replicate and/or measure a target nucleic acid strand from Yersinia pestis for pneumonic plague or severe acute respiratory syndrome coronavirus 2 (SARS-COV-2) for coronavirus disease 2019 (COVID-19), etc. Relative to qPCR, PCA may reduce amplification time (e.g., approximately 1/10th of qPCR time) for rapid testing. For instance, PCA may utilize relatively rapid heating and cooling cycles. In some examples of PCA, a heating cycle may be performed on the order of microseconds or milliseconds (e.g., 5 microseconds (us), 15 μs, 50 μs, 100 μs, 200 μs, 0.5 milliseconds (ms), 1 ms, 2 ms, etc.) and/or may heat a portion of a nucleic acid sample. In some examples of PCA, cooling to annealing and/or extension temperatures may occur on the order of seconds (e.g., 1, 2, 3, 4, 5, 6 seconds, etc.). In some examples, a complete cycle (for heating, cooling, and/or measurement) may be completed on the order of seconds (e.g., 4, 5, 6, 10 seconds, etc.). In some examples, a PCA amplification procedure may take a few minutes (e.g., 7, 10, 15, 20 minutes, etc.) to complete. As a trade-off, PCA measurements (e.g., curves) may show an exponential shape with more embedded noise relative to a sigmoid shape of qPCR measurements (e.g., curves). Due to the exponential shape and/or increased noise in PCA measurements, it can be difficult to achieve the same level of sensitivity and specificity of qPCR. In some approaches, detection thresholds are manually set by experienced technicians. Manually setting detection thresholds may suffer from subjectivity, update complexity, and/or relatively long time delay for use in new applications.

In some examples of the techniques described herein, RT-PCR may be utilized to amplify RNA. For instance, a reverse transcriptase (RT) technique may be utilized to detect RNA via PCA.

Some examples of the techniques described herein may help detect a target molecule in a substance (e.g., target nucleic acid strand in a nucleic acid sample) using data-driven approaches. Some examples of the techniques described herein may be performed without manual threshold setting and/or may be utilized for relatively fast model updating for new applications.

Some examples of the techniques described herein may utilize a machine learning model or models to detect a target molecule (e.g., target nucleic acid strand). A machine learning model is a structure that learns based on training. Examples of a machine learning model may include a regression model (e.g., regularized logistic regression models), a support vector machine (SVM), and an artificial neural network (e.g., deep neural networks, convolutional neural networks (CNNs), etc.). Training the machine learning model may include adjusting a weight or weights of the machine learning model. For example, a neural network may include a set of nodes, layers, and/or connections between nodes. The nodes, layers, and/or connections may have associated weights. The weights may be adjusted to train the neural network to perform a function, such as detecting a target molecule in a substance based on a plot of fluorescence measurements.

Some examples of the techniques described herein utilize a plot or plots of fluorescence measurements (e.g., PCA curve(s), qPCR curve(s), etc.). A plot is a graphical representation of data (e.g., fluorescence measurements). In some examples, a machine learning model (e.g., CNN) may be trained for target molecule (e.g., target nucleic acid strand) detection based on a plot or plots.

Throughout the drawings, similar reference numbers may designate similar or identical elements. When an element is referred to without a reference number, this may refer to the element generally, with and/or without limitation to any particular drawing or figure. In some examples, the drawings are not to scale and/or the size of some parts may be exaggerated to more clearly illustrate the example shown. Moreover, the drawings provide examples in accordance with the description. However, the description is not limited to the examples provided in the drawings.

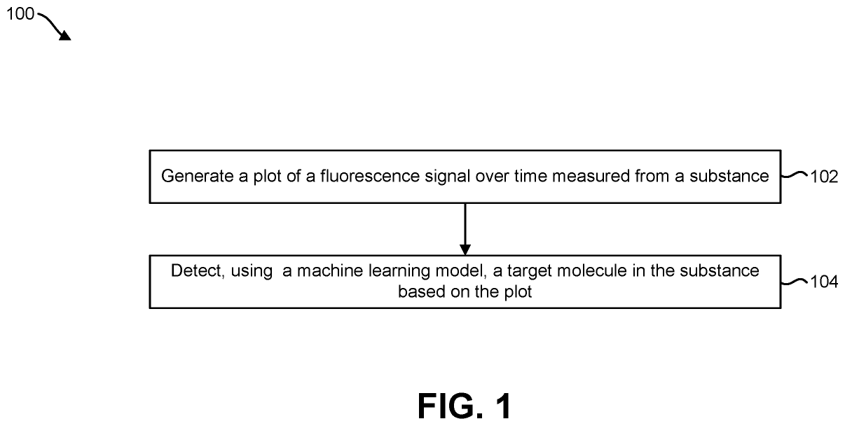

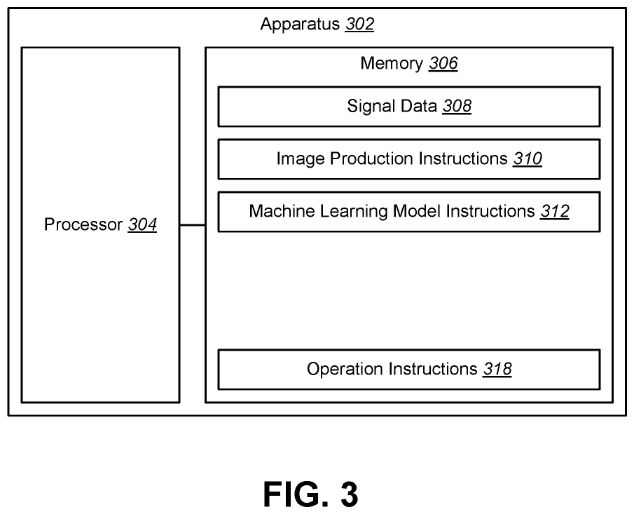

FIG. 1 is a flow diagram illustrating an example of a method 100 for target molecule detection. The method 100 and/or an element or elements of the method 100 may be performed by an apparatus (e.g., electronic device). For example, the method 100 may be performed by the apparatus 302 described in relation to FIG. 3.

The apparatus may generate 102 a plot of a fluorescence signal over time measured from a substance. For instance, a fluorescence signal may be measured over time from an amplification procedure of a substance (e.g., nucleic acid sample). In some examples, the apparatus may perform the amplification procedure and/or measure the fluorescence signal. In some examples, another device may perform the amplification procedure, measure the fluorescence signal, and/or send data indicating the fluorescence signal to the apparatus.

In some examples, the apparatus or another device may measure fluorescence signal strength and/or a fluorescence signal amplitude over time (e.g., over the amplification procedure). For instance, during the amplification procedure, the fluorescence signal may be measured at a series of time increments. In some examples, samples of the fluorescence signal may be measured in volts (V), in current (e.g., amperes (A)), in relative fluorescence units, or in other units. For instance, a light emitter may excite the substance (e.g., nucleic acid sample) during the amplification procedure. A light sensor may sense and/or measure the fluorescence signal produced by the light sensor when sensing light (e.g., fluorescence) emitted by the substance. For instance, the light sensor may measure the fluorescence signal as a voltage amplitude, current amplitude, or as another metric.

In some examples, the method 100 may include smoothing the fluorescence signal to produce a smoothed fluorescence signal. For instance, the apparatus may calculate the smoothed fluorescence signal by computing a moving average (e.g., sliding window average, weighted moving average, etc.) of the fluorescence signal, low-pass filtering the fluorescence signal, and/or performing curve fitting (e.g., least-squares curve fitting) on the fluorescence signal, etc. Smoothing the fluorescence signal may reduce high frequency noise. In some examples, generating 102 the plot of a fluorescence signal as described herein may be based on the raw fluorescence signal and/or based on the smoothed fluorescence signal.

In some examples, generating 102 the plot of the fluorescence signal over time may include generating a graphical representation of the fluorescence signal over time. For instance, the apparatus may generate a plot as an image where one dimension (e.g., vertical dimension, y dimension, etc.) of the image represents fluorescence amplitude and another dimension (e.g., horizontal dimension, x dimension, etc.) of the image represents time (in minutes or another unit, for instance). The fluorescence signal may be represented as a line and/or curve in the plot. In some examples, the plot may be expressed as an image. For instance, the plot may be expressed as pixel data depicting the fluorescence signal. In some examples, the plot may be a black and white image, a grayscale image, or a color image.

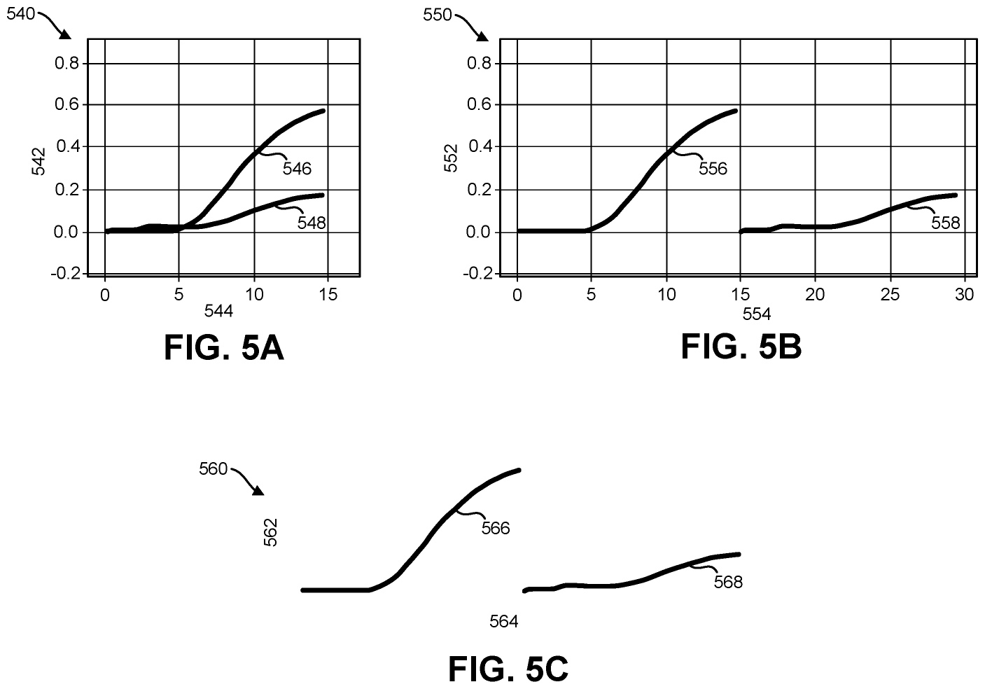

In some examples, the plot may include a scale. A scale is a marking or markings and/or value or values indicating a dimension or dimensions and/or a quantity or quantities. For instance, the plot may include a line indicating an axis or lines indicating axes (e.g., cartesian axes). In some examples, the axes may correspond to fluorescence and time. In some examples, the scale may include the axis or axes, mark or marks, and/or a number or numbers indicating a placement or mapping of samples of the fluorescence signal. In some examples, a plot may exclude a scale. For instance, a plot may not include an (e.g., may not include any) axis, mark, or number. Examples of plots are illustrated in FIGS. 5A, 5B, and 5C.

In some examples, the method 100 may include generating a second plot of a second fluorescence signal measured from the substance. For instance, the apparatus may generate a second plot corresponding to second samples of a second fluorescence signal measured during the amplification procedure of the substance. In some examples, the fluorescence signal may correspond to a first fluorophore (e.g., FAM or another fluorophore) and the second fluorescence signal may correspond to a second fluorophore (e.g., Cy5 or another fluorophore). For instance, the fluorescence signal may be measured using a first wavelength of light emitted into the substance and the second fluorescence signal may be measured using a second wavelength of light emitted into the substance. In some examples, any positive quantity of fluorescence signals may be measured and/or plotted in accordance with some examples of the techniques described herein. For instance, the method 100 may include generating a plurality of plots (e.g., 2, 3, 4, 6, 8, 10, 11, 16, 20, 32, etc., plots) of respective fluorescence signals (e.g., 2, 3, 4, 6, 8, 10, 11, 16, 20, 32, etc., fluorescence signals) measured from the substance. In some examples, each of the plots may correspond to a respective fluorescence signal corresponding to a respective fluorophore. In some examples, the plots may share a scale. In some examples, the plots may be sized according to the same scale (with or without the scale being included in the plots, for instance).

In some examples, the method 100 may include shifting the second plot to a spatially separate region. A spatially separate region is a region in a plot (e.g., image) where lines or curves representing fluorescence signals do not intersect. For instance, the apparatus may spatially shift the second fluorescence signal to avoid intersection(s) between (e.g., overlapping) lines or curves representing the fluorescence signals. Spatially separating the lines or curves may disambiguate, clarify, and/or disentangle the lines or curves, which may enhance target molecule detection in some examples. In some examples, the apparatus may produce an image by placing a first plot in a first region and a second plot in a spatially separate region from the first plot.

In some examples, the method 100 may include shifting the second plot along an axis. For instance, the apparatus may shift the second plot horizontally, vertically, and/or along another axis. In some examples, the apparatus may shift the second plot by adding a value to all samples of the second fluorescence signal. For instance, the axis may be a time axis. In some examples, the apparatus may shift the second plot by adding a value (e.g., 15 minutes or another value) to all of the samples of the second fluorescence signal. An example of adding a time shift to a second plot is illustrated in FIG. 5B.

In some examples, the apparatus may shift the second plot by adding a maximum value (with or without an offset, for instance) of the first fluorescence signal to the second fluorescence signal samples. For instance, the apparatus may add a maximum time value of the first fluorescence signal (e.g., 894 seconds) to all time values of the second fluorescence signal samples (with or without 1 second or another offset, for instance). In some examples, the apparatus may add a maximum fluorescence value of the first fluorescence signal (e.g., 0.58 V) to all fluorescence values of the second fluorescence signal samples (with or without 0.1 V or another offset, for instance). In some examples, the second plot may be shifted such that none of the sample values of the second plot overlap with a sample value of the first plot in a dimension or dimensions (e.g., such that any value on the x axis corresponds to a single value on the y axis).

In some examples, other approaches may be utilized to shift the second plot. For instance, the apparatus may shift the second plot until none of the pixels representing the second plot overlap with a pixel of the first plot. In some examples, a plurality of plots may be placed in spatially separate regions. For instance, a technique or techniques described herein may be utilized to shift (e.g., translate in a dimension or dimensions) a plot or plots of the plurality of plots to a spatially separate region or regions.

The apparatus may detect 104, using a machine learning model, a target molecule in the substance based on the plot. For instance, the apparatus may detect a target nucleic acid strand in the nucleic acid sample based on the plot. In some examples, the apparatus may input the plot to the machine learning model. The machine learning model may detect whether the substance includes the target molecule. For instance, the machine learning model may classify the substance based on the plot and/or may infer whether the substance includes the target molecule based on the plot. The machine learning model may be trained to detect whether the target molecule (e.g., nucleic acid strand) is in the substance (e.g., nucleic acid sample).

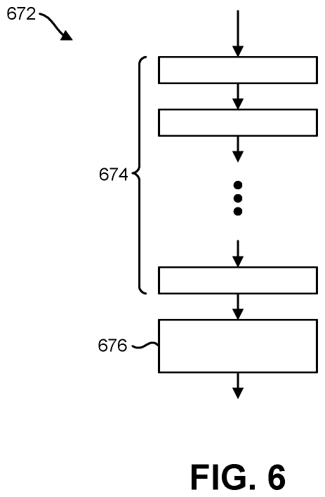

In some examples, the machine learning model may be trained with labeled plots. For example, the apparatus or another device may perform supervised training on the machine learning model. For instance, a training dataset may include plots labeled to indicate whether the plots correspond to a substance (e.g., nucleic acid sample) that included the target molecule (e.g., target nucleic acid strand). The weights of the machine learning model may be adjusted to reduce (e.g., minimize) classification error and/or to produce a decision boundary (e.g., decision hyperplane) that reduces (e.g., minimizes) misclassifications. In some examples, the machine learning model may be an artificial neural network (e.g., CNN), regression model, k-nearest neighbors model, or another machine learning model. In some examples, the machine learning model may be a CNN. For instance, the machine learning model may include a plurality of convolution layers and a fully connected classifier layer. An example of an architecture for the machine learning model is described in relation to FIG. 6.

Once the machine learning model is trained, the machine learning model may be executed to detect the target molecule (e.g., nucleic acid strand) in the substance (e.g., nucleic acid sample) based on the plot. For instance, the machine learning model may classify the plot to indicate whether the substance includes the target molecule. In some examples, detecting the target molecule may be based on multiple plots. For instance, detecting the target molecule may be further based on the second plot described above. In some examples, detecting the target molecule may be based on a plurality of plots (e.g., 2, 3, 4, 6, 8, 10, 11, 16, 32, etc., plots). For instance, an image may include a plurality of plots, where the image may be provided to a machine learning model (that has been trained on images with pluralities of plots, for example) to detect the target molecule.

In some examples, the apparatus may perform an operation based on the detection. For instance, the apparatus may output an indicator (e.g., symbol, word, message, color, text, tone, sound, and/or speech, etc.) indicating whether the target molecule was detected based on the plot. In some examples, the apparatus may send an indicator to another device indicating whether the target molecule was detected based on the plot(s). For instance, the apparatus may send a message (e.g., packet(s), email, text message, phone call, alert, etc.) to another device (e.g., computer, smartphone, tablet device, and/or server, etc.) indicating whether the target molecule was detected.

In some examples, the apparatus may perform the amplification procedure on the substance (e.g., nucleic acid sample). For instance, the apparatus may include a reaction chamber. A reaction chamber is a container and/or device that may be utilized to carry out a reaction (e.g., PCA or qPCR amplification procedure). In some examples, the reaction chamber may include a heating element (e.g., heating plate, heating coil, etc.). The apparatus may control the heating element to cyclically heat the substance in the reaction chamber to a target temperature or temperatures. In some examples, the reaction chamber may include a light emitter(s) and a light sensor(s). For instance, the apparatus may control the light emitter to cyclically emit light into the nucleic acid sample. The apparatus may take measurements from the light sensor. For instance, the apparatus may include an analog-to-digital converter (ADC) that samples voltages or currents taken from the light sensor. The measurements may be captured over a period as the fluorescence signal and/or as samples of a fluorescence signal. The fluorescence signal may be utilized to generate 102 the plot. In some examples, the amplification procedure is a PCA or qPCR amplification procedure.

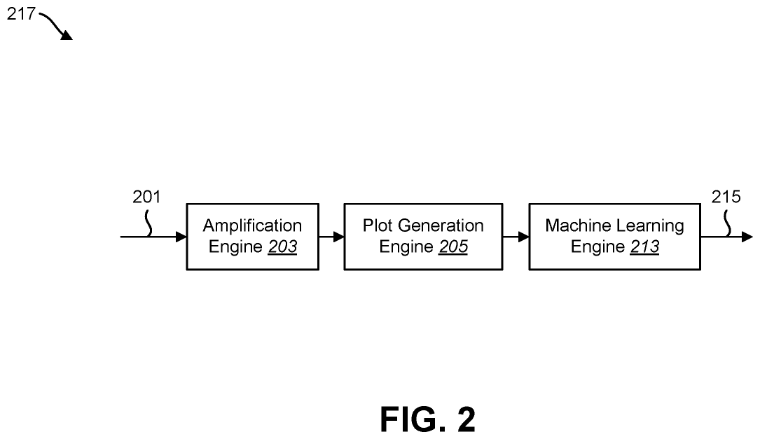

FIG. 2 is a block diagram illustrating an example of engines 217 that may be utilized in accordance with some examples of the techniques described herein. In some examples, an engine or engines of the engines 217 described in relation to FIG. 2 may be included in the apparatus 302 described in relation to FIG. 3. In some examples, a function or functions described in relation to any of FIGS. 1-6 may be performed by an engine or engines described in relation to FIG. 2. An engine or engines described in relation to FIG. 2 may be a device or devices, hardware (e.g., circuitry) and/or a combination of hardware and instructions (e.g., processor and instructions). The engines described in relation to FIG. 2 include an amplification engine 203, plot generation engine 205, and a machine learning engine 213. The engines 217 may be included in a same device or may be included in different devices in some examples. For instance, the amplification engine 203 may be included in a first device that may measure a fluorescence signal. The measured fluorescence signal may be provided to another device that includes the plot generation engine 205 and the machine learning engine 213. In other examples, the amplification engine 203, the plot generation engine 205, and the machine learning engine 213 may be included in one device.

In some examples of the techniques described herein, a substance 201 (e.g., nucleic acid sample) may be provided to the amplification engine 203. For instance, a technician may pipette the nucleic acid sample (with primer and fluorophore, for instance) into a reaction chamber of the amplification engine 203. The amplification engine 203 may perform an amplification procedure (e.g., PCA or qPCR) and measure a fluorescence signal(s) as described herein. The fluorescence signal(s) may be provided to the plot generation engine 205.

The plot generation engine 205 may generate a plot(s) of the fluorescence signal(s). For instance, the plot generation engine 205 may smooth the fluorescence signal(s) as described in relation to FIG. 1. The plot generation engine 205 may utilize the smoothed fluorescence signal(s) to produce a plot or plots. For instance, the plot generation engine 205 may generate a graphical representation(s) of the smoothed fluorescence signal(s). In some examples, the plot generation engine 205 may generate the plot(s) as described in relation to FIG. 1. For instance, the plot generation engine 205 may produce an indicator or indicators (e.g., points, line(s), curve(s), etc.) of the smoothed fluorescence signal samples in a graphical space (e.g., image space). In some examples, the plot generation engine 205 may determine the dimensions of the plot(s) based on the fluorescence signal samples. For instance, the plot generation engine 205 may select a plot dimension or dimensions to accommodate the fluorescence signal samples. In some examples, the plot generation engine 205 may utilize a static plot size. The plot(s) may be provided to the machine learning engine 213.

The machine learning engine 213 may determine, using a machine learning model, whether the substance 201 includes a target molecule based on the plot(s). For instance, the machine learning engine 213 may classify the substance 201 as including the target molecule (e.g., target nucleic acid strand) or not based on the plot(s) as described in relation to FIG. 1. The machine learning engine 213 may produce an indicator 215 (e.g., message, number, text, etc.) indicating whether the substance 201 includes a target molecule. For instance, the indicator 215 may be displayed, used to produce an output, and/or sent to another device to indicate whether the substance 201 (e.g., nucleic acid sample) includes the target molecule (e.g., nucleic acid strand).

FIG. 3 is a block diagram of an example of an apparatus 302 that may be used in molecule detection. The apparatus 302 may be a computing device, such as a personal computer, a server computer, a smartphone, a tablet computer, an electronic diagnostic device, an electronic testing device, a mobile testing device, a handheld electronic device, etc. The apparatus 302 may include and/or may be coupled to a processor 304 and/or a memory 306. The processor 304 may be in electronic communication with the memory 306. In some examples, the apparatus 302 may be in communication with (e.g., coupled to, have a communication link with) another device or devices (e.g., reaction chamber, amplification device, PCA device, server, computer, smartphone, tablet device, etc.). In some examples, the apparatus 302 may be an example of a computer. In some examples, the apparatus 302 may be an example of a medical testing device. The apparatus 302 may include additional components (not shown) and/or some of the components described herein may be removed and/or modified without departing from the scope of this disclosure.

In some examples, the apparatus 302 may perform a technique or techniques (e.g., measurement, plotting, and/or detection, etc.) described herein without sending data to and/or receiving data from another device (e.g., a cloud server, an edge device, a networked device, etc.). For instance, the apparatus 302 may be a local medical testing device and/or computer, where a communication bus and/or network interface is not used to send and/or receive data pertaining to some examples of the techniques described herein (e.g., measurement, plotting, and/or detection). In some examples, a technique or techniques (e.g., measurement, plotting, and/or detection, etc.) described herein may be performed in conjunction with sending data to and/or receiving data from another device (e.g., a cloud server, an edge device, a networked device, etc.). For instance, the apparatus 302 may be a local medical testing device and/or computer that sends fluorescence signal(s) and/or plot(s)) to a cloud server to perform a technique or techniques described herein (e.g., plotting and/or detection) and receives data (e.g., test results) from the cloud server. In some examples, the apparatus 302 may be a cloud server or edge device that receives data (e.g., fluorescence signal(s) and/or plot(s)) from another device, performs plotting and/or detection, and sends data (e.g., results) to another device (e.g., endpoint node).

The processor 304 may be any of a central processing unit (CPU), a semiconductor-based microprocessor, graphics processing unit (GPU), field-programmable gate array (FPGA), an application-specific integrated circuit (ASIC), and/or other hardware device suitable for retrieval and execution of instructions stored in the memory 306. The processor 304 may fetch, decode, and/or execute instructions (e.g., image production instructions 310, machine learning model instructions 312, and/or operation instructions 318) stored in the memory 306. In some examples, the processor 304 may include an electronic circuit or circuits that include electronic components for performing a functionality or functionalities of the instructions (e.g., image production instructions 310, machine learning model instructions 312, and/or operation instructions 318). In some examples, the processor 304 may perform one, some, or all of the functions, operations, elements, methods, etc., described in relation to one, some, or all of FIGS. 1-6.

The memory 306 may be any electronic, magnetic, optical, or other physical storage device that contains or stores electronic information (e.g., instructions and/or data). Thus, the memory 306 may be, for example, Random Access Memory (RAM), Electrically Erasable Programmable Read-Only Memory (EEPROM), a storage device, an optical disc, and the like. In some examples, the memory 306 may be a non-transitory tangible machine-readable storage medium, where the term “non-transitory” does not encompass transitory propagating signals.

In some examples, the apparatus 302 may also include a data store (not shown) on which the processor 304 may store information. The data store may be volatile and/or non-volatile memory, such as Dynamic Random-Access Memory (DRAM), EEPROM, magnetoresistive random-access memory (MRAM), phase change RAM (PCRAM), memristor, flash memory, and the like. In some examples, the memory 306 may be included in the data store. In some examples, the memory 306 may be separate from the data store. In some approaches, the data store may store similar instructions and/or data as that stored by the memory 306. For example, the data store may be non-volatile memory and the memory 306 may be volatile memory.

In some examples, the apparatus 302 may include an input/output interface (not shown) through which the processor 304 may communicate with an external device or devices (not shown), for instance, to send and/or receive data. The input/output interface may include hardware and/or machine-readable instructions to enable the processor 304 to communicate with the external device or devices. The input/output interface may enable a wired and/or wireless connection to the external device or devices. In some examples, the input/output interface may further include a network interface card and/or may also include hardware and/or machine-readable instructions to enable the processor 304 to communicate with various input and/or output devices, such as a keyboard, a mouse, a display, touch screen, another apparatus, electronic device, computing device, etc., through which a user may input instructions into the apparatus 302. In some examples, the apparatus 302 may receive signal data 308 from an external device or devices (e.g., reaction chamber, testing device, etc.). For instance, the apparatus 302 may receive signal data 308 that indicates a fluorescence signal measured from an amplification procedure performed by a separate reaction chamber.

In some examples, the memory 306 may store signal data 308. Some examples of signal data 308 include data representing a fluorescence signal(s) measured from an amplification procedure. The signal data 308 may be measured by the apparatus 302 and/or received from another device. For instance, the apparatus 302 may include a reaction chamber in some examples. The apparatus 302 (e.g., processor 304) may control the reaction chamber to perform an amplification procedure (on a substance or nucleic acid sample, for instance). The apparatus 302 (e.g., processor 304) may measure a fluorescence signal (from the reaction chamber, for instance). For example, the processor 304 may control a reaction chamber to cyclically heat the nucleic acid sample, to emit light into a nucleic acid sample, and to measure fluorescence emitted from the nucleic acid sample. The measured fluorescence may be stored in the signal data 308 as a fluorescence signal. In some examples, the processor 304 may control the reaction chamber (or chambers) to determine multiple fluorescence signals, which may be stored in the signal data 308.

In some examples, the processor 304 may measure a first fluorescence signal and a second fluorescence signal. For instance, the processor 304 may measure samples of a first fluorescence signal by controlling a light source to emit light at a first wavelength for a first fluorophore and by controlling a light detector to detect fluorescence for the first fluorophore. In some examples, the processor 304 may also measure samples of a second fluorescence signal by controlling the same or a different light source to emit light at a second wavelength for a second fluorophore and by controlling the same or a different light detector to detect fluorescence for the second fluorophore.

The memory 306 may store image production instructions 310. The processor 304 may execute the image production instructions 310 to determine a plot or plots based on the fluorescence signal(s) represented by the signal data 308. In some examples, the processor 304 may produce an image including a first plot of a first fluorescence signal and a second plot of a second fluorescence signal from an amplification procedure of a nucleic acid sample as described in relation to FIG. 1 and/or FIG. 2.

In some examples, the processor 304 may execute the image production instructions 310 to produce the image by placing the first plot in a first region and the second plot in a spatially separate second region from the first plot. In some examples, producing the image may be performed as described in relation to FIG. 1, and/or FIG. 2.

The memory 306 may store machine learning model instructions 312. The processor 304 may execute the machine learning model instructions 312 to determine, using a machine learning model, whether the nucleic acid sample includes a nucleic acid strand based on the image. For instance, the processor 304 may execute a machine learning model that is trained based on a plot or plots to detect the target nucleic acid strand. In some examples, the machine learning model may detect the target nucleic acid strand in the nucleic acid sample as described in relation to FIG. 1 and/or FIG. 2.

In some examples, the processor 304 may execute the operation instructions 318 to perform an operation. For example, the apparatus 302 may perform an operation based on the determination of whether the nucleic acid sample includes the target nucleic acid strand. For instance, the apparatus 302 may output an indicator (e.g., symbol, word, message, color, text, tone, sound, and/or speech, etc.) indicating whether the target nucleic acid strand was detected based on the plot(s). In some examples, the apparatus 302 may send an indicator to another device indicating whether the target nucleic acid strand was detected based on the plot(s). For instance, the apparatus 302 may be a server that receives a fluorescence signal from another device and provides a testing web service. The apparatus 302 may send a message (e.g., packet(s), email, text message, phone call, alert, etc.) to another device (e.g., computer, smartphone, tablet device, and/or server, etc.) indicating whether the target nucleic acid strand was detected. For instance, the apparatus 302 may send a message to a requesting device indicating whether the target nucleic acid strand was detected. In some examples, the apparatus 302 may send a message to another device (e.g., server) to report a number of cases in which the target nucleic acid strand was detected.

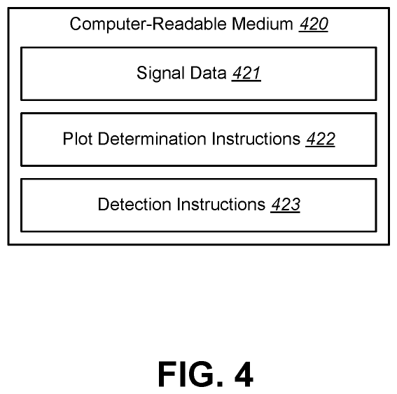

FIG. 4 is a block diagram illustrating an example of a computer-readable medium 420 for molecule detection. The computer-readable medium 420 may be a non-transitory, tangible computer-readable medium 420. The computer-readable medium 420 may be, for example, RAM, EEPROM, a storage device, an optical disc, and/or the like. In some examples, the computer-readable medium 420 may be volatile and/or non-volatile memory, such as DRAM, EEPROM, MRAM, PCRAM, memristor, flash memory, and/or the like. In some examples, the memory 306 described in connection with FIG. 3 may be an example of the computer-readable medium 420 described in connection with FIG. 4.

The computer-readable medium 420 may include data (e.g., information and/or instructions). For example, the computer-readable medium 420 may include signal data 421, plot determination instructions 422, and/or detection instructions 423.

In some examples, the computer-readable medium 420 may store signal data 421. Some examples of signal data 421 include data representing a fluorescence signal or signals, sample data, etc. For instance, the signal data 421 may represent a fluorescence signal or signals measured from an amplification (e.g., PCA, qPCR, etc.) procedure.

In some examples, the plot determination instructions 422 may be instructions when executed cause a processor of an electronic device to determine a plot of a fluorescence signal measured from a PCA procedure. In some examples, determining the plot may be accomplished as described in relation to FIG. 1, FIG. 2, and/or FIG. 3.

In some examples, the detection instructions 423 may be instructions when executed cause the processor to execute a machine learning model to detect a target nucleic acid strand in a nucleic acid sample based on the plot. In some examples, detecting the target nucleic acid strand may be accomplished as described in relation to FIG. 1, FIG. 2, and/or FIG. 3.

In some examples, the machine learning model may include a plurality of convolution layers and a fully connected classifier layer. An example of an architecture for a machine learning model is described in relation to FIG. 6.

In some examples, the computer-readable medium 420 may include instructions when executed cause the processor to train the machine learning model. For instance, the processor may input a training dataset into the machine learning model and evaluate a loss function and/or objective function to adjust weights of the machine learning model and reduce loss and/or misclassification errors.

In some examples, the machine learning model may be trained based on minority oversampling. For instance, minority oversampling may be utilized for cases where a training dataset is imbalanced (where a minority class in the training dataset underrepresents the frequency that the class occurs in real-world data, for example). In some examples, the processor may perform minority oversampling by selecting a random example from the minority class. The processor may find k nearest neighbors to the selected example. The processor may select one of the nearest neighbors randomly. The processor may create a synthetic example at a randomly selected point between the two examples in feature space. The processor may repeat this oversampling procedure to create more samples (until a threshold amount of samples is created, for instance). For example, more samples may be created until the minority class is represented approximately at a frequency that reflects real-world occurrence of the class.

FIG. 5A is a graph 540 illustrating examples of a first plot 546 of a first fluorescence signal and a second plot 548 of a second fluorescence signal. For example, the graph 540 illustrates the first plot 546 and the second plot 548 in fluorescence signal amplitude 542 (in volts) over time 544 (in minutes). The first plot 546 and the second plot 548 may represent fluorescence signals produced from an amplification procedure (e.g., PCA, qPCR, etc.). In the example of FIG. 5A, the first plot 546 corresponds to a FAM fluorophore (e.g., E gene channel), and the second plot 548 corresponds to a Cy5 fluorophore (e.g., internal control channel).

In some examples, first plot 546 may indicate a sample measurement and the second plot 548 may indicate an internal process control measurement, which may be utilized to ensure that a reaction chamber is functioning correctly. The graph 540 illustrates a positive result (where the target molecule is included in the substance, for instance). In the example of FIG. 5A, the first plot 546 provides an indication that a substance is positive. In some examples, a substance may include the target molecule when a corresponding fluorescence signal plot has a positive slope and a final value that is greater than 0.2, while a substance may not include the target molecule when a corresponding fluorescence signal plot has a nearly flat slope and a final value that is less than 0.2.

Some examples of the techniques described herein may utilize a machine learning model (e.g., CNN, deep learning model, etc.) to perform image recognition. For instance, an apparatus may generate an image of a plot or plots (e.g., the first plot 546 for a FAM fluorophore and the second plot 548 for a Cy5 fluorophore). The machine learning model may be trained to recognize positive cases and negative cases based on the image of the plot(s).

FIG. 5B is a graph 550 illustrating examples of a first plot 556 of a first fluorescence signal and a second plot 558 of a second fluorescence signal. For example, the graph 550 illustrates the first plot 556 and the second plot 558 in fluorescence signal amplitude 552 (in volts) over time 554 (in minutes). The first plot 556 and the second plot 558 may represent fluorescence signals produced from an amplification procedure (e.g., PCA, qPCR, etc.). In the example of FIG. 5B, the first plot 556 corresponds to a FAM fluorophore, and the second plot 558 corresponds to a Cy5 fluorophore. The first plot 556 of FIG. 5B may correspond to the first plot 546 of FIG. 5A and the second plot 558 of FIG. 5B may correspond to the second plot 548 of FIG. 5A.

In some examples of the techniques described herein, plots may be separated in an image. In the example of FIG. 5B, the second plot 558 is shifted to a spatially separate region from a region of the first plot 556. In this example, the second plot 558 has been shifted along the time axis to separate the second plot 558 from the first plot 556. For instance, the first plot 556 that corresponds to a FAM fluorophore may be placed next to the second plot 558 that corresponds to a Cy5 fluorophore. In this example, a scale is included with the first plot 556 and the second plot 558.

FIG. 5C is a graph 560 illustrating examples of a first plot 566 of a first fluorescence signal and a second plot 568 of a second fluorescence signal. For example, the graph 560 illustrates the first plot 566 and the second plot 568 in fluorescence signal amplitude 562 (in volts) over time 564 (in minutes). The first plot 566 and the second plot 568 may represent fluorescence signals produced from an amplification procedure (e.g., PCA, qPCR, etc.). In the example of FIG. 5C, the first plot 566 corresponds to a FAM fluorophore, and the second plot 568 corresponds to a Cy5 fluorophore. The first plot 566 of FIG. 5C may correspond to the first plot 546 of FIG. 5A and the second plot 568 of FIG. 5C may correspond to the second plot 548 of FIG. 5A.

In some examples of the techniques described herein, a plot scale may be excluded from an image. In the example of FIG. 5C, the image excludes a scale. For instance, an image of the first plot 566 and the second plot 568 may be provided to a machine learning model without a scale in some approaches. In some examples, the exclusion or inclusion of a scale may not have a significant impact on detection accuracy.

FIG. 6 is a diagram illustrating an example of an architecture 672 of a machine learning model that may be utilized in some examples of the techniques described herein. The architecture 672 may include a plurality of convolution layers 674 (e.g., N convolution layers) and a fully connected classifier layer 676. In some examples, the architecture 672 may be utilized to perform convolution on an input image that includes a plot or plots of a fluorescence signal or signals and to classify the input image as indicating a target molecule (e.g., target nucleic acid strand) in a substance (e.g., nucleic acid sample) or not.

Some examples of the techniques described herein may enhance a limit of detection and/or detection sensitivity. A limit of detection may be a quantity and/or concentration (e.g., lowest quantity and/or concentration, minimum quantity and/or concentration, etc.) at which a target molecule may be detected (with a degree of certainty and/or reliability, for example). Some approaches to detection may detect a virus with 95% accuracy at a limit of detection of 1000 copies per milliliter (copies/ml) of substance. Some examples of the techniques described herein may reduce the limit of detection. For instance, some examples of the techniques may detect a virus with 95% accuracy with a limit of detection of about 500 copies/ml. Accordingly, some examples of the techniques described herein may provide increased detection accuracy (at lower limit of detection values, for instance). For instance, some examples of the techniques described herein may provide increased accuracy at a limit of detection relative to other approaches. Increased detection accuracy may help to reduce the spread of disease in some cases.

As used herein, the term “and/or” may mean an item or items. For example, the phrase “A, B, and/or C” may mean any of: A (without B and C), B (without A and C), C (without A and B), A and B (but not C), B and C (but not A), A and C (but not B), or all of A, B, and C.

While various examples of systems and methods are described herein, the systems and methods are not limited to the examples. Variations of the examples described herein may be within the scope of the disclosure. For example, operations, functions, aspects, or elements of the examples described herein may be omitted or combined.

1. A method, comprising:
generating a plot of a fluorescence signal over time measured from a substance; and
detecting, using a machine learning model, a target molecule in the substance based on the plot.

2. The method of claim 1, further comprising generating a second plot of a second fluorescence signal measured from the substance, wherein detecting the target molecule is further based on the second plot.

3. The method of claim 2, further comprising shifting the second plot to a spatially separate region.

4. The method of claim 2, further comprising shifting the second plot along an axis.

5. The method of claim 4, wherein the axis is a time axis.

6. The method of claim 2, wherein the fluorescence signal corresponds to a first fluorophore and the second fluorescence signal corresponds to a second fluorophore.

7. The method of claim 1, wherein the plot excludes a scale.

8. The method of claim 1, wherein the machine learning model is a convolutional neural network.

9. The method of claim 1, further comprising generating a plurality of plots of respective fluorescence signals measured from the substance, wherein detecting the target molecule is further based on the plurality of plots.

10. An apparatus, comprising:
a memory;
a processor in electronic communication with the memory, wherein the processor is to:
produce an image comprising a first plot of a first fluorescence signal and a second plot of a second fluorescence signal from an amplification procedure of a nucleic acid sample; and
determine, using a machine learning model, whether the nucleic acid sample includes a nucleic acid strand based on the image.

11. The apparatus of claim 10, wherein the processor is to:
produce the image by placing the first plot in a first region and the second plot in a spatially separate second region from the first plot.

12. The apparatus of claim 11, further comprising:
a reaction chamber, wherein the processor is to:
control the reaction chamber to perform the amplification procedure; and
measure the first fluorescence signal and the second fluorescence signal.

13. A non-transitory tangible computer-readable medium comprising instructions when executed cause a processor of an electronic device to:
determine a plot of a fluorescence signal measured from a pulse-controlled amplification (PCA) procedure; and
execute a machine learning model to detect a target nucleic acid strand in a nucleic acid sample based on the plot.

14. The non-transitory tangible computer-readable medium of claim 13, wherein the machine learning model includes a plurality of convolution layers and a fully connected classifier layer.

15. The non-transitory tangible computer-readable medium of claim 13, wherein the machine learning model is trained based on minority oversampling.
